(19) United States
(12) Patent Application Publication
(10) Pub. No.: US 2026/0036702 A1
(43) Pub. Date: Feb. 5, 2026

(54) RADIATION DOES DISTRIBUTION MEASUREMENT DEVICE AND RADIATION IRRADIATION APPARATUS

(71) Applicant: The University of Tokyo, Tokyo (JP)

(72) Inventor: Takeshi OHTA, Tokyo (JP)

(73) Assignee: The University of Tokyo, Tokyo (JP)

(21) Appl. No.: 19/354,942

(22) Filed: Oct. 10, 2025

Related U.S. Application Data
(63) Continuation of application No. PCT/JP2024/014872, filed on Apr. 12, 2024.

(30) Foreign Application Priority Data
Apr. 12, 2023 (JP) 2023-064982

(51) Int. Cl. G01T 1/20; A61N 5/10

(52) U.S. Cl. CPC G01T 1/2002; A61N 5/1071

(57) ABSTRACT

A radiation dose distribution measurement device of a radiotherapy apparatus capable of rapidly identifying a detection plane of a scintillator, having high precision and high repeatability, and easily expanding the scope of application. The radiation dose distribution measurement device includes a housing, a scintillator module which is removably attached to the housing, and emits fluorescence when radiation is irradiated from the radiation irradiation apparatus, and a camera which is removably attached to the housing, and shoots the fluorescence from the scintillator module. The scintillator module includes a scintillator held between transparent resin plates respectively located on both main faces of the scintillator, each transparent resin plate having a thickness of 1 cm to 10 cm, and markers are respectively formed along four corners of the transparent resin plate located on the side of the camera for identifying a detection plane of the scintillator.

### TECHNICAL FIELD

The present disclosure relates to a radiation dose distribution measurement device of a radiation irradiation apparatus. 3

### BACKGROUND ART

A radiotherapy apparatus is a therapeutic apparatus which irradiates radiation from outside of the body of a patient toward tumors in the body to destroy cancer cells forming the tumor. On the other hand, it is necessary to minimize the influence of the radiation on normal cells around the tumors. Therefore, in order to irradiate a radiation dose sufficient to destroy the cancer cells, while minimizing the radiation dose to the normal cells, the radiation dose and the cumulative radiation dose to the tumors and therearound are verified before treatment.

Namely, CT (Computed Tomography) images, etc., are used as images for a treatment plan, and a radiation dose distribution and a cumulative radiation dose distribution (simulation images) are created on the images by a radiation treatment planning system as the treatment plan. Next, the radiotherapy apparatus is actually operated, the cumulative radiation dose distribution (actual measurement image) is acquired by the radiation dose distribution measurement device, and whether the simulation image matches the actual measurement image is checked, to thereby perform quality control.

For example, Patent Document 1 and Patent Document 2 disclose an absorbed dose distribution measurement apparatus for actual measurement of the cumulative radiation dose distribution, in which a scintillation fiber block or a flat-plate plastic scintillator emits light in proportion to the absorption amount of the irradiated radiation, an image measuring device (camera) measures the light intensity distribution at the end face (detection plane) of the scintillation fiber block or the flat-plate plastic scintillator, and based on this, a three-dimensional or two-dimensional absorbed dose distribution is measured.

### PRIOR ARTS

Patent Document 1: Japanese Unexamined Patent Publication (Kokai) No. 2002-267754
Patent Document 2: Japanese Unexamined Patent Publication (Kokai) No. 2003-240858

### SUMMARY

However, according to the conventional absorbed dose distribution measurement apparatus, identifying the actual measurement plane (detection plane) of the scintillator is complicated. Further, based on the premise that the camera and the scintillator are fixed to the apparatus, the combination of the camera and the scintillator cannot be changed depending on the purpose of use, and thus, expanding the scope of application is difficult.

One of the objectives of the present disclosure is to provide a radiation dose distribution measurement device, for a radiation irradiation apparatus such as a radiotherapy apparatus, capable of rapidly identifying the detection plane of the scintillator, achieving high precision and high repeatability, and easily expanding the scope of application.

In order to attain the above objective, present disclosure includes the following aspects.

[1] A radiation dose distribution measurement device of a radiation irradiation apparatus, comprising: a housing, a scintillator module which is removably attached to the housing, and emits fluorescence when radiation is irradiated from the radiation irradiation apparatus, and a camera which is removably attached to the housing, and shoots the fluorescence from the scintillator module, wherein the scintillator module comprises a scintillator held between transparent resin plates respectively located on both main faces of the scintillator, each transparent resin plate having a thickness of 1 cm to 10 cm, and markers are respectively formed along four corners of the transparent resin plate located on the side of the camera for identifying a detection plane of the scintillator.

[2] A radiation dose distribution measurement device of a radiation irradiation apparatus according to [1], wherein, the marker is configured to receive fluorescence and Cherenkov light from the scintillator module, and to emit light in the direction of the camera.

[3] A radiation dose distribution measurement device of a radiation irradiation apparatus according to [1], wherein the transparent resin plate has a thickness of 1.5 cm to 2.5 cm.

[4] A radiation dose distribution measurement device of a radiation irradiation apparatus according to [1], wherein the scintillator has a thickness of 1.0 mm to 10.0 mm.

[5] A radiation dose distribution measurement device of a radiation irradiation apparatus according to [1], wherein an anti-glare film is provided between the scintillator and the transparent resin plate located on the camera side, or an anti-glare layer is formed on a contact surface of the scintillator and the transparent resin plate located on the camera side.

[6] A radiation dose distribution measurement device of a radiation irradiation apparatus according to any one of [1] to [5], wherein the scintillator emits blue fluorescence.

[7] A radiation dose distribution measurement device of a radiation irradiation apparatus according to any one of [1] to [5], wherein the scintillator emits red fluorescence.

[8] A radiation dose distribution measurement device of a radiation irradiation apparatus according to any one of [1] to [5] further comprising an absolute dosimeter for calibration which measures dose of radiation irradiated to the scintillator module.

[9] A radiation dose distribution measurement device of a radiation irradiation apparatus according to any one of [1] to [5] further comprising a light incident window formed on a side of the scintillator module opposite to the side of the camera, and a light-reduction film held between the scintillator of the scintillator module and the transparent resin plate located on the side opposite to the side of the camera, wherein the camera receives laser beams that are irradiated from a laser beam irradiation device for positioning which has been set in advance, and are incident from the incident window through the scintillator module, to display a laser beam image and a reference point image for position verification on a display device.

[10] A radiation dose distribution measurement device of a radiation irradiation apparatus according to [9], wherein radiation for position verification is irradiated from the radiation irradiation apparatus to the scintillator module, the scintillator module emits fluorescence, and the reference point image is an image captured by shooting the fluorescence by the camera.

[11] A radiation dose distribution measurement device of a radiation irradiation apparatus according to [9], wherein the camera receives visible light through the scintillator module, the visible light indicating an irradiation range of the radiation which is irradiated from the radiation irradiation apparatus, and is incident from the incident window, to display an image representing the radiation irradiation range, and the reference point image for position verification on the display device.

According to the present disclosure, an actual measurement plane (detection plane) can be rapidly identified by position recognition of a predetermined marker. Thereby, a radiation dose distribution measurement device, for a radiation irradiation apparatus, can be provided, by which even if the optical axis is somewhat moved from a predetermined position, due to the removal, installation, etc., of the camera, the influence to the radiation dose distribution measurement can be suppressed, high precision and high repeatability can be achieved, scope of application can be easily expanded.

### BRIEF DESCRIPTION OF DRAWINGS

FIG. 1 is a cross-sectional view showing a configuration example of a radiation dose distribution measurement device of a radiotherapy apparatus according to the present aspect.

FIG. 2 is a perspective view showing a scintillator module according to the present aspect.

FIG. 3 is a cross-sectional view showing another configuration example of a radiation dose distribution measurement device of a radiotherapy apparatus.

FIG. 4A, FIG. 4B, and FIG. 4C are explanatory views explaining a method for identifying a detection plane of a scintillator according to the present aspect.

FIG. 5A is a view showing an example of an actual measurement image according to the present aspect.

FIG. 5B is a view showing an example of a simulation image according to the present aspect.

FIG. 6A and FIG. 6B are schematic explanatory views of gamma pass analysis.

FIG. 7 is a view showing still another configuration example of a radiation dose distribution measurement device of a radiotherapy apparatus according to the present aspect.

FIG. 8 is a view showing an example of a laser beam image output from a camera, a radiation irradiation range image during treatment, and a reference point image.

FIG. 9 is a perspective view showing another configuration example of a scintillator module according to the present aspect.

### ASPECTS OF DISCLOSURE

Hereinbelow, aspects of the present disclosure (hereinbelow, referred to as aspects) will be explained with reference to the drawings.

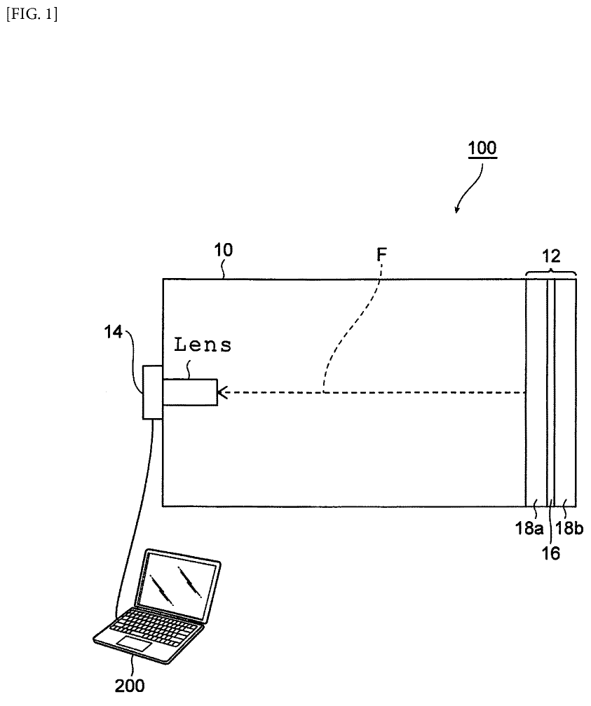

FIG. 1 is a cross-sectional view showing a configuration example of a radiation dose distribution measurement device 100 of a radiation irradiation apparatus having a function of irradiating radiation, such as a radiotherapy apparatus, etc., according to an aspect. In FIG. 1, the radiation dose distribution measurement device 100 comprises a scintillator module 12 which is removably attached to a housing 10 and emits fluorescence F having an intensity corresponding to the radiation dose irradiated from a radiotherapy apparatus (not shown), and a camera 14 which is removably attached to the housing 10 and shoots the fluorescence F emitted from the scintillator module 12. The housing 10 can be, for example, a hollow quadrangular prism having, for example, a square cross-section. Materials for the housing 10 can be, for example, an acrylic resin such as polymethylmethacrylate (PMMA), expanded polystyrene, gypsum board, Medium Density Fiberboard (MDF), Carbon Fiber Reinforced Plastics (CFRP), ABS resin, and the like. By using such materials, the housing can be made lighter (amount of substance can be reduced) to the extent that the radiation irradiated for medical treatment is not disturbed. Preferably, the wall of the housing 10 has a hollow structure so that the housing is made further lighter. The details of the scintillator module 12 will be described below. In the aspect described below, an example using a radiotherapy apparatus as a radiation irradiation apparatus will be explained.

The camera 14 can be, for example, a CMOS camera, etc., although the camera 14 is not limited thereto as far as the camera can shoot the fluorescence F from the scintillator module 12 and can generate an actual measurement image. Preferably, the camera 14 is arranged so that its optical axis is perpendicular to one of the main faces of the scintillator module 12. According to the present aspect, the camera 14 is used for shooting the fluorescence F, and thus, the resolution of the captured image is determined depending on the resolution of the camera 14, but the resolution can be increased to the submillimeter level. The image data, which is the output from the camera 14, is imported to a computer 200 through an appropriate interface (USB, wired or wireless LAN, etc.), and then, below-mentioned processes such as identification of the detection plane of the scintillator 16, gamma pass analysis between the actual measurement image and the simulation image, and the like, are executed.

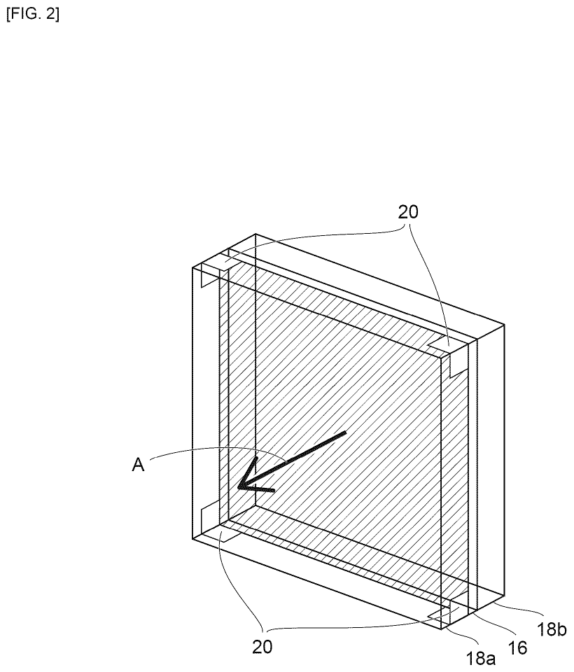

FIG. 2 shows a perspective view of a configuration example of the scintillator module 12. In FIG. 2, the scintillator module 12 comprises a flat-plate scintillator 16 which is held between transparent resin plates 18a and 18b respectively provided on both main faces of the scintillator 16. In the example of FIG. 2, the transparent resin plates 18a and 18b are drawn as transparent, and the scintillator 16 can be seen through the transparent resin plate 18a. Here, in FIG. 2, the camera 14 is located at a predetermined position in the direction of the arrow A (the arrow set on the optical axis of the camera 14), and the transparent resin plate 18a is arranged so that one of the main faces of the transparent resin plate 18a is in contact with the main face, on the camera 14 side, of the scintillator 16. The transparent resin plate 18b is arranged so that one of the main faces of the transparent resin plate 18b is in contact with the main face, on the side opposite to the camera 14 side, of the scintillator 16. Further, the material for the transparent resin plate 18b is not limited to the transparent resin. For example, using an opaque resin such as a black resin, etc., to block the light from the back side (the side opposite to the camera 14 side) of the scintillator 16 is preferable. Also, a light-blocking film can be provided between the scintillator 16 and the transparent resin plate 18b. In the example of FIG. 2, when the scintillator 16 is viewed from the point on the optical axis of the camera 14, the main face of the scintillator 16 has a square shape, but the shape is not limited thereto. For example, the main face can be rectangular, circular, etc. Further, the main face of the scintillator 16 can be referred to as a detection plane.

Each of the transparent resin plates 18a and 18b has a thickness of preferably 1 cm to 10 cm. Since the thickness is 1 cm to 10 cm, a sufficient amount of secondary electrons can be generated in the transparent resin plates 18a and 18b by entered radiation, to thereby increase the amount of light emitted from the scintillator. In this case, in order to measure a dose distribution close to the dose distribution in the patient's body, the larger thickness (for example, 10 cm) is preferable. However, when the thickness, the weight is increases, and thus, handling becomes difficult. In view of these points, each of the transparent resin plates 18a and 18b has a thickness of more preferably 1.5 cm to 2.5 cm, and still more preferably 2.0 cm.

In order to hold the scintillator 16 between the transparent resin plates 18a and 18b, the transparent resin plates 18a and 18b can be adhered to the scintillator 16 by using an optical adhesive, but the adhesion can be done without using the optical adhesive. When the scintillator 16 is held between the transparent resin plates 18a and 18b without using the optical adhesive, air is present between the scintillator 16 and each of the transparent resin plates 18a and 18b. In this case, a warpage occurs particularly in the transparent resin plate 18a, and a moire pattern (newton ring) may be generated in the actual measurement image. In order prevent this, providing an anti-glare film between the transparent resin plate 18a and the scintillator 16, or forming an anti-glare layer on a surface of the transparent resin plate 18a which is in contact with the scintillator 16. For the anti-glare film or the anti-glare layer, a conventionally known one can be used.

A material for the transparent resin plates 18a and 18b is a resin composed of carbon, hydrogen, and oxygen, which may further contain nitrogen. Further, the resin for the material preferably has a density close to the density of water. This is because, since the radiation therapy is applied to the human body, the resin composed of elements which composes the human body, and having a density closer to that of the human body is preferable so as to easily replicate the dose distribution in the patient's body, and to precisely measure the radiation dose distribution.

Examples of the material for the transparent resin plates 18a and 18b include an acrylic resin, polycarbonate etc. Among them, the acrylic resin is the most preferable. As for the acrylic resin, an acrylic ester polymer or a methacrylic ester polymer is preferable, and polymethylmethacrylate (PMMA) is more preferable.

The scintillator 16 is not limited as far as the scintillator emits fluorescence when the radiation is irradiated from the radiotherapy apparatus. For example, a blue scintillator which emits blue fluorescence when the radiation is irradiated, a red scintillator which emits red fluorescence when the radiation is irradiated, etc., can be used.

The shape of the scintillator 16 is not limited as far as the shape is a plate shape. For example, the main face can be a square, and the thickness thereof is preferably 1.0 mm to 10.0 mm, and more preferably 1.0 mm to 2.5 mm. Here, the smaller the thickness, the higher the resolution on the slice direction (resolution in the direction perpendicular to the main face), but the lower the amount of light. Further, when the resolution in the slice direction increases, corresponding to this resolution, the below-mentioned resolution of the simulation image should be increased, resulting in increasing the calculation time for the simulation image producing process. On the other hand, the larger the thickness, the higher the amount of light, but the lower the resolution in the slice direction. In view of the above, the thickness of the scintillator 16 is determined in the above range, and thickness is more preferably 2.0 mm.

As shown in FIG. 2, the transparent resin plate 18a is arranged on the camera 14 side of the scintillator 16, and is provided, at each of the four corners thereof, with a marker 20 for identifying the detection plane (main face) of the scintillator 16. The marker 20 has a structure to receive the fluorescence from the scintillator 16 (scintillator module 12) and the Cherenkov light from the transparent resin plate 18a, and to emit light in the direction of the camera 14. Examples of such a structure include: a fine uneven structure such as frosted glass, a reflection structure formed by adhering a reflective tape or applying a reflective paint, a fluorescence emission structure formed by fluorescent coating, and the like. In FIG. 2, the marker 20 has a flat L-shape extending along each of the four corners of the transparent resin plate 18a, the depth of the marker 20 being approximately half the width of the transparent resin plate 18a, and the cross-section of the marker 20 parallel to the main face of the transparent resin plate 18a being L-shape (the shape of the Japanese style quotation mark). In this case, each line segment forming the L-shape has a length approximately the same as the depth mentioned above. When the marker 20 having the above-mentioned shape is shot by the camera 14, due to the angle of view of the camera 14, the image of the light-emission face of the marker 20 (the plane formed along the four corners) appears as a thin L-shaped line.

Further, the shape of the marker 20 does not have to be the above-mentioned shape, and can be any shapes such as a quadrangle, a triangle, a circle, a line segment, and the like, as far as the markers are formed on the four corners of the main face of the transparent resin plate 18a, and the positions of the four corners can be recognized by emitting light in the direction of the camera 14.

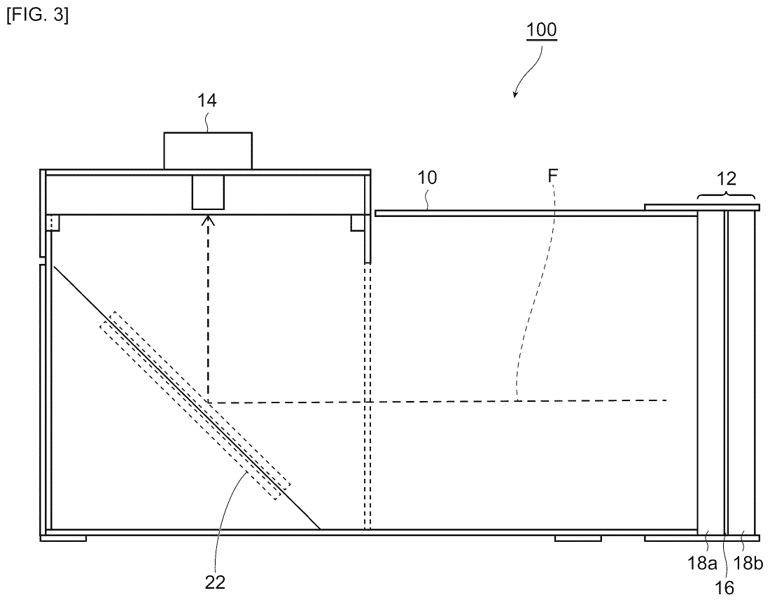

FIG. 3 is a cross-sectional view showing another configuration example of the radiation dose distribution measurement device 100 of the radiotherapy apparatus according to the present aspect. According to the configuration example shown in FIG. 3, the fluorescence F is emitted from the scintillator module 12, the traveling direction of the fluorescence F is changed by a reflective mirror 22, and the fluorescence F is shot by the camera 14 which is removably attached to the housing 10 at a position where the extension in the travelling direction of the fluorescence F intersects with the housing 10. Preferably, the reflective mirror 22 is a front surface mirror produced by forming, by vapor-deposition, a light reflection layer on the light incident face of the glass plate or the resin plate, and applying mirror processing thereto. A reflective mirror in which mirror processing is applied to the face of the glass plate or the resin plate opposite to the light incident face (back surface mirror) is not preferable, because a ghost may be generated by multiple reflections, and the Cherenkov light may be generated when the radiation passes through the transparent resin, resulting in disturbing the observation of the scintillation light, and lowering the measurement precision of the radiation dose distribution. As shown in FIG. 3, by appropriately changing the traveling direction of the fluorescence F emitted from the scintillator module 12, using the reflective mirror 22, etc., the camera 14 can be attached at any desired position.

As mentioned above, in the radiation dose distribution measurement device 100 of the radiotherapy apparatus according to the present aspect, the scintillator module 12 and the camera 14 are removably attached to the housing 10. Therefore, depending on the purpose of use, the attachment positions of the scintillator module 12 and the camera 14 can be determined in any combination, such that the positions are changed therebetween. Thus, the scope of application of the radiation dose distribution measurement device 100 can be expanded.

Further, as shown in FIG. 2, the markers 20 are formed at the four corners of the transparent resin plate 18a, and the detection plane (main face) of the scintillator 16 can be identified on the basis of the positions of the markers 20 in the actual measurement image captured by the camera 14. Here, the identifying the detection plane refers to determining the range of the detection plane of the scintillator 16 on the actual measurement image. Thereby, even in the case that the radiation is irradiated only on a part of the scintillator 16, and only a part of the scintillator emits the fluorescence (the part to which no radiation is irradiated is darkened), the range of the detection plane of the scintillator 16 can be recognized.

The gap between the markers 20 is determined depending on the size of the scintillator 16, and thus, the rotation, distortion of the image of the scintillator 16 can be corrected on the basis of the coordinates (any desired coordinate system can be used) of the markers 20 in the actual measurement image. Therefore, when the scintillator module 12 and the camera 14 are replaced and the position of the optical axis is changed along with the removal, attachment, etc., the position can be corrected by the above correction, and thus, it is not necessary to precisely determine the positional relationship therebetween once again. Accordingly, replacement, etc., of the scintillator module 12 and the camera 14 can be performed rapidly.

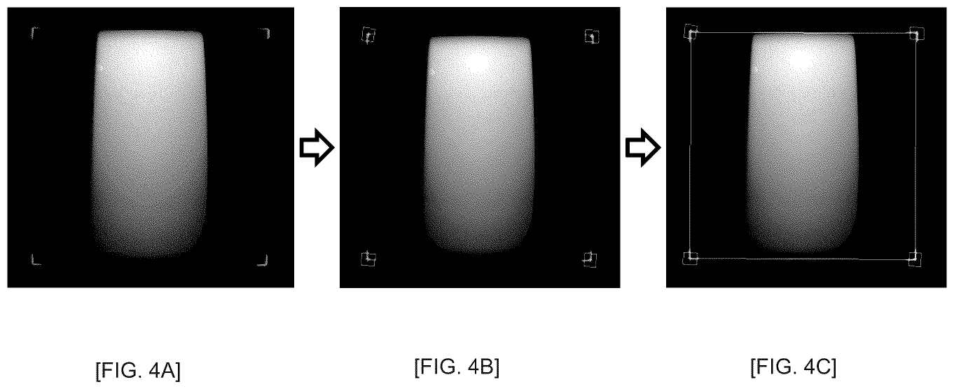

FIG. 4A, FIG. 4B, and FIG. 4C are explanatory views showing a method for identifying the detection plane of the scintillator 16. FIG. 4A shows the actual measurement image of the detection plane of the scintillator 16 captured by the camera 14, in which the markers 20 indicating the four corners of the scintillator 16 are shown, each marker 20 being shown in L-shape. The shape of the marker 20 is registered in advance in an identification processing program which operates on the computer 200, and thus, the positions of the markers 20 can be recognized from the actual measurement image. In FIG. 4B, a square is made around each of the L-shaped markers 20. This indicates that the identification processing program recognizes the markers 20. Next, the identification processing program determines the range of the detection plane on the basis of the positions of the four markers 20. Here, as mentioned above, the rotation, distortion, etc., of the image of the scintillator 16 is corrected, and thereafter, the range of the detection plane is determined. FIG. 4C shows the range of the detection plane identified by four lines connecting the four markers 20.

### <Method for Using Radiation Dose Distribution Measurement Device 100 of Radiotherapy Apparatus According to Present Aspect>

First, as for a therapeutic plan, an image of a cumulative radiation dose distribution is created by a radiation therapy planning apparatus, as a simulation image, using a previously obtained CT image of a patient. Here, the cumulative radiation dose distribution refers to a distribution obtained by integrating the radiation dose distribution at each time point (moment) during the operation of the radiotherapy apparatus, with respect to any selected operation time (for example, the total operation time) of the radiotherapy apparatus.

Next, in order that a cumulative radiation dose distribution, same as the cumulative radiation dose distribution shown in the above-mentioned simulation image, can be obtained as a result of the actual radiation irradiation, instruction information which instructs irradiation conditions is input to a control device (program) which controls the operations of the radiotherapy apparatus. Thereafter, the radiation dose distribution measurement device 100 is positioned and installed at the isocenter which is the center of the rotation mechanism of the radiotherapy apparatus. The radiotherapy apparatus is operated, and radiation is irradiated in accordance with the instruction information which has been set on the basis of the therapeutic plan previously made for the radiation dose distribution measurement device 100. The irradiated radiation is captured by the scintillator module 12 of the radiation dose distribution measurement device 100, and the fluorescence F emitted from the scintillator module 12 is shot and imaged by the camera 14, to thereby obtain the actual measurement image.

In this case, the fluorescence F can be shot successively while the radiotherapy apparatus is in operation, and thus, the radiation irradiation state can be observed in the chronological order by the actual measurement image.

When the fluorescence F is shot, the markers 20 are also shot by the camera 14, the markers 20 being respectively formed at the four corners of the transparent resin plate 18a to receive the fluorescence and Cherenkov light and to emit light in the direction of the camera 14. As described above, the position of the scintillator module 12 (scintillator 16) on the treatment table is determined at the isocenter of the radiotherapy apparatus, and thus, the absolute coordinates (the coordinates in the coordinate system that the radiation therapy planning apparatus has) of the markers 20 in the actual measurement image can be determined in advance, the actual measurement image having been corrected by correcting the rotation, distortion of the image of the scintillator 16 in accordance with needs. Accordingly, a gamma pass analysis program running on the computer 200 compares the detection plane of the scintillator 16 identified (that is to say, the desired range is determined) on the simulation image in the absolute coordinate system of the radiation therapy planning apparatus, with the detection plane of the scintillator 16 identified on the basis of the markers 20 on the actual measurement image, and the degree of match therebetween is confirmed by the gamma pass analysis mentioned below. As mentioned above, thanks to the markers 20, the comparison between the actual measurement image and the simulation image can be performed easily and rapidly.

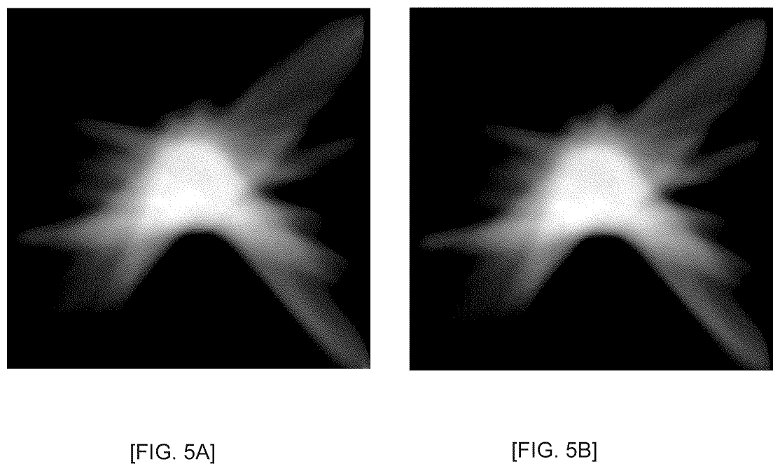

FIG. 5A and FIG. 5B show examples of the actual measurement image and the simulation image. FIG. 5A is an example of the actual measurement image, and FIG. 5B is an example of the simulation image. Here, both of FIG. 5A and FIG. 5B are images of the cumulative radiation dose distribution.

When there is a high degree of match between the actual measurement image shown in FIG. 5A and the simulation image shown in FIG. 5B, it can be judged that operating the radiotherapy apparatus (therapy by radiation irradiation) is appropriate in the therapeutic plan. Here, the degree of match between the actual measurement image and the simulation image is judged by, for example, the gamma pass analysis.

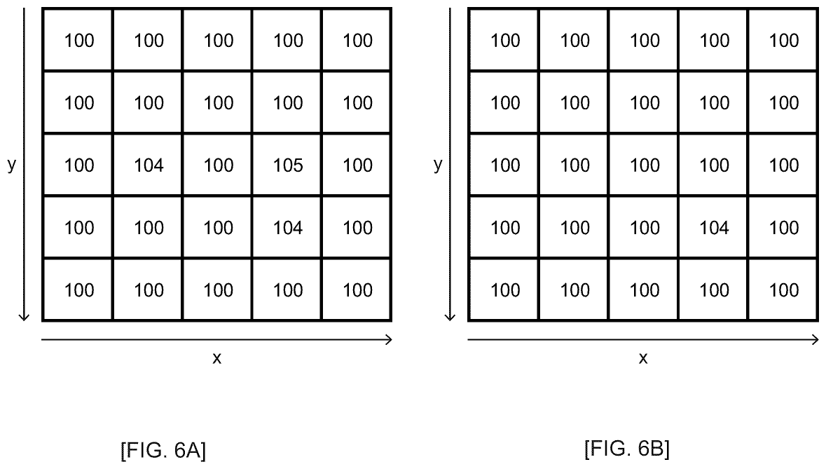

FIG. 6A and FIG. 6B show schematic explanatory view of the gamma pass analysis. FIG. 6A shows the actual measurement image, FIG. 6B shows a reference image. Here, the reference image is an image used for analyzing the degree of match with the actual measurement image. For example, the reference image can be a simulation image, or can be an actual measurement image. Further, each of the actual measurement image shown in FIG. 6A and the reference image shown in FIG. 6B is divided into square regions having the same size. The position of each square region is represented by the coordinate (x, y) using the x-axis and y-axis shown in FIG. 6A and FIG. 6B. Here, the same x-axis and y-axis are set in the actual measurement image and the reference image, the axes being coordinate axes in the x-axis direction and the y-axis direction with the sequential numerical values for the square regions. Further, the numerical value described in each square represents each pixel value, that is, the value representing the amount of light emission of the scintillator 16 in each square region.

In case of the radiotherapy apparatus, the actual measurement image and the reference image are divided into square regions, one side of the square region being, for example, 1 mm, and the threshold value of the gamma pass analysis is set to 2 mm 3%, that is to say, with respect to a relevant square region on the actual measurement image, a corresponding square region (where the (x, y) coordinate matches) on the reference image is determined, and if there is a square region having a pixel value difference of 3% or less among the square regions, which satisfy that the distance from the center of each of the square regions to the center of the corresponding square region is 2 mm or less, the relevant square region on the actual measurement image is determined as passed.

In the example shown in FIG. 6A and FIG. 6B, the square region (x, y)=(4, 3) in the actual measurement image has a pixel value of 105, whereas the square region (x, y)=(4, 4) in the reference image has a pixel value of 104, the square region (x, y)=(4, 4) being located within 2 mm from the corresponding square region (x, y)=(4, 3) in the reference image. Here, the difference between the pixel values is less than 1%, and thus, the square region (x, y)=(4, 3) is determined as passed. Further, the square region (x, y)=(4, 4) in the actual measurement image has a pixel value of 104, whereas the square region (x, y)=(4, 4) in the reference image has a pixel value of 104, the square region (x, y)=(4, 4) being located within 2 mm from the corresponding square region (x, y)=(4, 4) in the reference image. Here, the difference between the pixel values is 0%, and thus, the square region (x, y)=(4, 4) is determined as passed. On the other hand, the square region (x, y)=(2, 3) in the actual measurement image has a pixel value of 104, whereas all the square regions located within 2 mm from the corresponding square region (x, y)=(2, 3) in the reference image have a pixel value 100. Here, the difference between the pixel values is 4%, and thus, the square region (x, y)=(2, 3) is determined as failed.

The above-mentioned gamma pass analysis is performed for the 25 square regions shown in FIG. 6A, and as a result, 24/25 are passed, and thus, as an analysis result, the pass rate is 96%. In case of the radiotherapy apparatus, in general, if the gamma pass analysis is performed at 2 mm 3%, and the pass rate is 95% or more, the degree of match between the actual measurement image and the reference image is determined as high.

### <Red Scintillator>

As explained above regarding FIG. 2, for the scintillator 16, not only the blue scintillator emitting blue fluorescence, but also the red scintillator emitting red fluorescence can be used.

The transparent resin plates 18a and 18b which hold the scintillator 16 therebetween, may be yellowish discolored (yellowing) due to deterioration over time caused by the radiation from the radiotherapy apparatus. In case that a blue scintillator is used for the scintillator 16, if the transparent resin plate 18 is yellowing, the blue wavelength component is absorbed, resulting in reducing the amount of light. Therefore, the preciseness of the actual measurement image shot by the camera 14 is lowered, and the credibility of the cumulative radiation dose distribution represented by the actual measurement image is lost.

On the other hand, in case of the fluorescence having a longer wavelength than that of blue (for example, a wavelength of 500 nm or more, such as red, etc.), even if the transparent resin plate 18a is yellowing, the wavelength component absorbed thereby is small, and thus, the decrease in preciseness of the actual measurement image can be suppressed. Accordingly, as a modified example of the present aspect, a red scintillator is used for the scintillator 16. Thereby, the credibility of the cumulative radiation dose distribution represented by the actual measurement image is increased, and the reliability of the radiation therapy is increased.

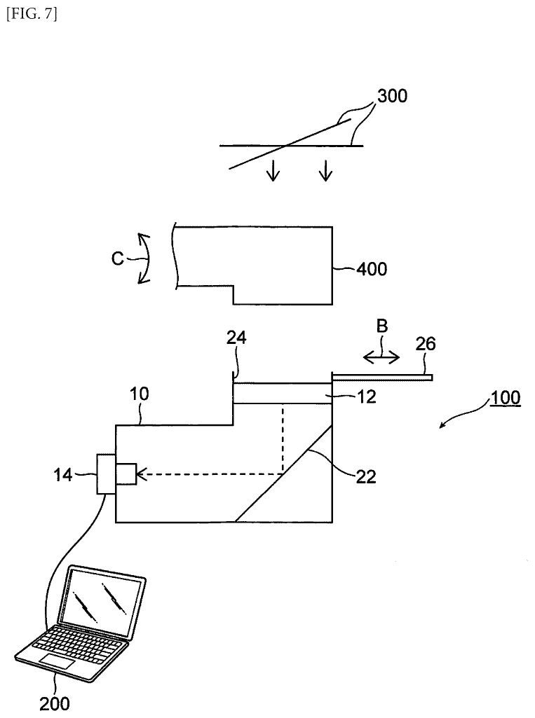

FIG. 7 shows still another configuration example of the radiation dose distribution measurement device 100 of the radiotherapy apparatus according to the present aspect. For the elements same as those FIG. 3, the same numerals are assigned and explanation therefor is omitted. In FIG. 7, the housing 10 has an opening at one side of the scintillator module 12, the side being opposite to the side of the camera 14 (the outer side of the housing 10), and an incident window 24 thorough which the light enters is formed at the opening.

The scintillator module 12 is integrally formed with the part where the incident window 24 is formed, and is configured to be exchangeable with the position where the camera 14 is attached. Preferably, the incident window 24 is provided with an opaque sliding lid 26 sliding, for example, in the direction of the arrow B to open/close the incident window 24. The type of the lid 26 is not limited to this.

In FIG. 7, laser beams 300 for positioning are irradiated by an appropriate laser beam irradiation device, from the ceiling of the room in which the radiation dose distribution measurement device 100 is located. The laser beams for positioning are output as linear laser beams perpendicularly intersecting with each other. The laser beams including the intersections enter the incident window 24 of the radiation dose distribution measurement device 100. On the basis of the intersections of straight lines indicated by the laser beams, and the position of the center of gravity in the radiation dose distribution, whether the geometric position of the radiation irradiation apparatus, such as a radiotherapy apparatus, etc., is appropriate, can be verified. The camera 14 receives the laser beams entering from the incident window 24 through the scintillator module 12, and outputs laser beam image data. The output laser beam image data is imported to the computer 200, and is displayed as a laser beam image for position verification, on an appropriate display device, such as a display, etc., of the computer 200.

Further, in FIG. 7, the radiation for position verification and visible lights indicating the radiation irradiation range during the treatment, are irradiated from the radiotherapy apparatus 400 as the radiation irradiation apparatus toward the incident window 24 of the radiation dose distribution measurement device 100. Here, the radiation for position verification indicates the position where the radiation is actually irradiated during the treatment, with an irradiation plane of preferably, 1 cm×1 cm, the size of which is not limited thereto.

When the visible light indicating the radiation irradiation range during the treatment enters from the incident window 24, the camera 14 receives the visible light entering from the incident window 24 through the scintillator module 12, and outputs visible light image data. The output visible light image data is imported to the computer 200, and is displayed as an image of the radiation irradiation range during the treatment, on an appropriate display device, such as a display, etc., of the computer 200. Here, the position of the center of gravity in the radiation dose distribution can be appropriately obtained by image processing of the image of the radiation irradiation range.

Further, when the radiation for position verification is irradiated from the radiotherapy apparatus 400, due to this radiation, fluorescence is emitted from the scintillator module 12, and the fluorescence is shot by the camera 14. The output from the camera 14 at this time is used for image data at the reference point for position verification. The image data at the reference point is imported in the computer 200, and is displayed as a reference point image, on an appropriate display device such as a display, etc., of the computer 200.

When the laser beams 300 are irradiated, the radiotherapy apparatus 400 is in the way, and thus, is moved, for example, in the arrow C direction (from the back to the front of the paper) by rotational movement, etc.

FIG. 8 shows examples of the laser beam image, the radiation irradiation range image during treatment, and the reference point image, output from the camera 14. In FIG. 8, a display screen 28 of the appropriate display device such as a display, etc., of the computer 200 displays the above images at positions on the screen corresponding to the incident positions from the incident window 24. In the example shown in FIG. 8, the laser beam images R are shown by perpendicularly intersecting straight lines, the radiation irradiation range image F (hereinbelow, referred to as the irradiation range image F) is shown by a rectangular range, and the reference point image P is shown by a rectangular range smaller than the irradiation range image F. The reference point image P is displayed on the same display screen 28 as where the laser beam image R and/or the irradiation range image F are displayed, and thus, on the basis of the mutual positional relationship, positions of the radiation dose distribution measurement device 100 and the radiation irradiation range during the treatment can be confirmed, that is, the deviation from the position where the radiation is actually irradiated during treatment, can be confirmed.

In an example shown in FIG. 8, the reference point image P is slightly deviated to the upper right on the display screen 28, relative to the laser beam images R and the irradiation range image F. Therefore, prior to the radiation therapy, the position of the radiation dose distribution measurement device 100 and the radiation irradiation range during the treatment can be correctly adjusted.

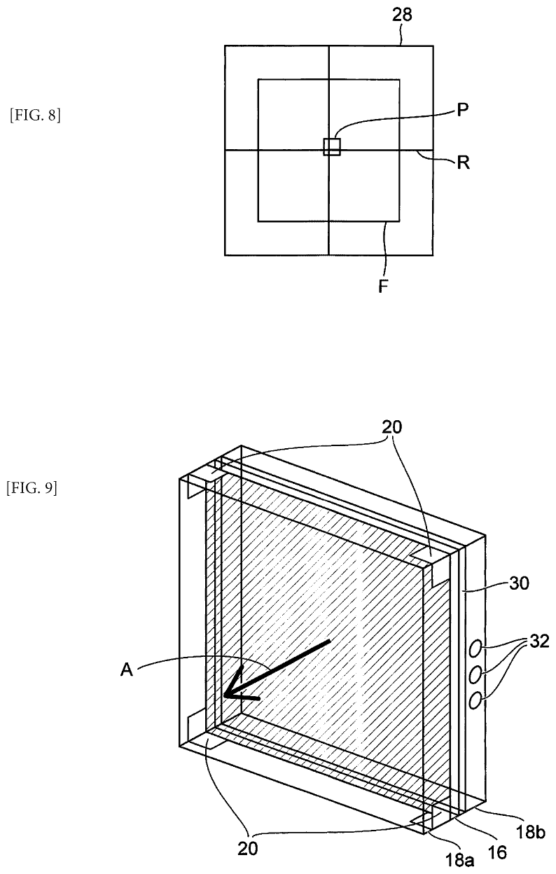

FIG. 9 shows a perspective view of another configuration example of the scintillator module 12. For the elements same as those shown in FIG. 2, the same numerals are assigned, and the explanation therefor is omitted. In FIG. 9, a light-reduction film 30 is held between the scintillator 16 and the transparent resin plate 18b located on a side of the scintillator 16 opposite to the side of the camera 14. When the laser beams and the visible light representing the radiation irradiation range transmit through the scintillator module 12, the light-reduction film 30 reduces the intensity of the light. Accordingly, the high intensity laser beam and the high intensity visible light can be prevented from entering the camera 14, and thus, shooting by the camera 14 can be correctly done.

Further, the transparent resin plate 18b located on the side of the scintillator module 12 opposite to the side of the camera 14 shown in FIG. 9, is provided with a port 32 to which an absolute dosimeter for calibration is to be inserted, the absolute dosimeter measuring the absolute value of the radiation dose irradiated to the scintillator module 12. On the basis of the absolute value of the radiation dose measured by the absolute dosimeter inserted in the port 32, the intensity of the fluorescence emitted from the scintillator 16 and the radiation dose can be calibrated. In the example shown in FIG. 9, there are three ports 32 to which the absolute dosimeter is to be inserted, which indicates that the absolute dosimeter can be inserted to any one of the three ports. It is not necessary to arrange three absolute dosimeters. Further, the number of the absolute dosimeter arrangement positions (the number of ports 32) is not limited to three, and the arrangement position can be appropriately set. Further, when the absolute dosimeter is not inserted, a dummy made of a resin is inserted in the port 32.

### EXPLANATION ON NUMERALS

10 housing, 12 scintillator module, 14 camera, 16 scintillator, 18a, 18b transparent resin plate, 20 marker, 22 reflective mirror, 24 incident window, 26 lid, 28 display screen, 30 light-reduction film, 32 port, 100 radiation dose distribution measurement device, 200 computer, 300 laser beam, 400 radiotherapy apparatus

1. A radiation dose distribution measurement device of a radiation irradiation apparatus, comprising:
a housing,
a scintillator module which is removably attached to the housing, and emits fluorescence when radiation is irradiated from the radiation irradiation apparatus, and
a camera which is removably attached to the housing, and shoots the fluorescence from the scintillator module, wherein
the scintillator module comprises a scintillator held between transparent resin plates respectively located on both main faces of the scintillator, each transparent resin plate having a thickness of 1 cm to 10 cm, and
markers are respectively formed along corners of the transparent resin plate located on the side of the camera for identifying a detection plane of the scintillator.

2. A radiation dose distribution measurement device of a radiation irradiation apparatus according to claim 1, wherein,
the marker is configured to receive fluorescence and Cherenkov light from the scintillator module, and to emit light in the direction of the camera.

3. A radiation dose distribution measurement device of a radiation irradiation apparatus according to claim 1, wherein the transparent resin plate has a thickness of 1.5 cm to 2.5 cm.

4. A radiation dose distribution measurement device of a radiation irradiation apparatus according to claim 1, wherein the scintillator has a thickness of 1.0 mm to 10.0 mm.

5. A radiation dose distribution measurement device of a radiation irradiation apparatus according to claim 1, wherein an anti-glare film is provided between the scintillator and the transparent resin plate located on the camera side, or an anti-glare layer is formed on a contact surface of the scintillator and the transparent resin plate located on the camera side.

6. A radiation dose distribution measurement device of a radiation irradiation apparatus according to claim 1, wherein the scintillator emits blue fluorescence.

7. A radiation dose distribution measurement device of a radiation irradiation apparatus according to claim 1, wherein the scintillator emits red fluorescence.

8. A radiation dose distribution measurement device of a radiation irradiation apparatus according to claim 1 further comprising an absolute dosimeter for calibration which measures dose of radiation irradiated to the scintillator module.

9. A radiation dose distribution measurement device of a radiation irradiation apparatus according to claim 1, further comprising
a light incident window formed on a side of the scintillator module opposite to the side of the camera, and
a light-reduction film held between the scintillator of the scintillator module and the transparent resin plate located on the side opposite to the side of the camera, wherein
the camera receives laser beams that are irradiated from a laser beam irradiation device for positioning which has been set in advance, and are incident from the incident window
through the scintillator module, to display a laser beam image and a reference point image for position verification on a display device.

10. A radiation dose distribution measurement device of a radiation irradiation apparatus according to claim 9, wherein radiation for position verification is irradiated from the radiation irradiation apparatus to the scintillator module, the scintillator module emits fluorescence, and the reference point image is an image captured by shooting the fluorescence by the camera.

11. A radiation dose distribution measurement device of a radiation irradiation apparatus according to claim 9, wherein the camera receives visible light through the scintillator module, the visible light indicating an irradiation range of the radiation which is irradiated from the radiation irradiation apparatus, and is incident from the incident window, to display an image representing the radiation irradiation range, and the reference point image for position verification on the display device.
